(19) United States
(12) Patent Application Publication
(10) Pub. No.: US 2023/0355086 A1
(43) Pub. Date: Nov. 9, 2023

(54) COMBINATION TOOTHBRUSH AND MIRROR DEVICE FOR DENTAL EXAMINATION

(71) Applicant: DR. BARNEY PARADIGMS, LC, Sandy, UT (US)

(72) Inventor: Barney Todd Olsen, Sandy, UT (US)

(21) Appl. No.: 18/131,272

(22) Filed: Apr. 5, 2023

Related U.S. Application Data
(60) Provisional application No. 63/338,558, filed on May 5, 2022.

(51) Int. Cl. A61B 1/247; A46B 9/04

(52) U.S. Cl. CPC A61B 1/247; A46B 9/04; A46B 2200/1066

(57) ABSTRACT

Disclosed is a combination toothbrush and mirror device configured to be used in dental examinations. The device contains a handle, shank, and head with bristles being on the front surface and a mirror being on the back surface. The mirror may be embedded, selectively detachable or attached using a tilt mechanism allowing the mirror to be held in a particular orientation by resistance or a fastening element.

### CROSS-REFERENCE TO RELATED APPLICATIONS

This application claims the benefit of and priority to U.S. Provisional Application No. 63/338,558, filed May 5, 2022, which is incorporated herein by reference in its entirety.

### BACKGROUND

Dentists and orthodontists commonly use mirrors during dental examinations to visualize patients' teeth. The mirror is normally attached to an elongated member which must be inserted into the patient's mouth. The mirror is then moved around in different orientations to visualize the teeth from multiple viewpoints.

Normally, visualizing teeth is straightforward; however, children and patients with special needs do not always like having objects placed in their mouths. Even simple objects like the mirror described above can cause discomfort and/or agitation, sometimes to the point where the visualization of the teeth is no longer possible. This makes the teeth visualization process difficult and sometimes impossible for dentists and caregivers.

This issue has resulted in subpar dental care for children and special needs patients. In some cases, dental examinations are not performed to the extent they should, causing dental issues to be overlooked or missed. Other times, dental examinations are stopped prematurely, and the mouth is not properly examined. This causes stress to the examiner/dentist, the patient, and caregiver(s), which could result in the patient not continuing to receive regular dental examinations.

In sum, using mirrors attached to an elongated member have caused problems in the dental examination process for children and patients with special needs. This makes dental examinations difficult for dentists, orthodontists, and caregivers, which furthermore results in improper dental care for children and patients with special needs. Accordingly, there is a long felt and ongoing need for improved dental examination devices to visualize teeth for children and patients with special needs.

### BRIEF SUMMARY

The present disclosure relates to a dental examination device which includes a toothbrush coupled with a mirror. This solves problems related to dental examinations for children and special needs children who do not like unknown objects such as a mirror placed in their mouth. The device described herein uses a toothbrush, which children and special needs patients are likely to be familiar with, as a distraction for such patients, while the mirror can be used to visualize the mouth and teeth. In particular, it has been surprisingly found that the brushing motion of the toothbrush provides a familiar distraction to the patient while the associated mirror allows the examination to take place.

Multiple embodiments are described to couple the mirror to the dental examination device. In some embodiments, the mirror is a circular shape. Other embodiments may include other rounded and polygonal shaped mirrors. One embodiment includes attaching the mirror using an adhesive. A second embodiment includes embedding the mirror into the dental examination device. A third embodiment includes the mirror being attached to a selectively detachable device which is then coupled to the mirror. Examples of the selectively detachable devices include a friction fit cap that keeps the bristles exposed, an attachable element that is configured to secure around the head of the dental examination device while keeping the bristles exposed, a hook attached to the mirror that goes around the shank of the dental examination device, or an elongated member where one end is attached to the mirror and the other end is attached to the shank or handle of the dental examination device.

Another embodiment includes the mirror being coupled with the dental examination device by using a tilt mechanism. The tilt mechanism allows the mirror to move along the latitudinal and longitudinal axis of the dental examination device. In one embodiment, the tilt mechanism holds the mirror in a particular orientation by using resistance. Another embodiment includes a fastening element attached to the tilt mechanism. The fastening element could be a screw, push-pull locking element, or other known fastening element in the art known to allow movement of the mirror and then hold the mirror in a particular orientation.

In use, the dental examination device is placed in a patient's mouth. The front surface of the device includes a brushing surface which is used to brush the teeth while the back surface of the device includes a mirror which is used to visualize the teeth. The device can be manually moved in the mouth to visualize different areas. In certain embodiments, the user may move the mirror to change the orientation. The mirror can then be held in that orientation before inserting the mirror into the mouth to ensure visualization from multiple angles.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an example of a dental examination device with a handle, shank, and head with bristles on the front surface and a mirror coupled to the back surface;

FIG. 2 illustrates the head of the dental examination device;

FIG. 3 illustrates a dental examination device with a rectangular mirror coupled to the back surface of the head;

FIG. 4 illustrates a dental examination device where the mirror is coupled to the back surface of the head using an adhesive;

FIG. 5 illustrates a dental examination device with the mirror embedded in the back surface of the head;

FIGS. 6 and 7 illustrates a dental examination device with a mirror coupled to the back surface by a friction fit cap with a front view of the head of the dental examination device with an example of a fiction fitted cap;

FIGS. 8 and 9 illustrates a side and front view of a dental examination device with a mirror attached to the back surface of the head by a cap configured to secure around the head while keeping the bristles exposed;

FIG. 10 illustrates a dental examination device with a mirror attached to the shank by a hook;

FIG. 11 illustrates a dental examination device with an elongated member attached on one end to a mirror and on the other end to the handle;

FIG. 12 illustrates a dental examination device with a mirror attached to the back surface using a tilt mechanism;

FIGS. 13 and 14 illustrate a dental examination device with a mirror attached by a tilt mechanism and the mirror held in different orientations; and

FIGS. 15 and 16 illustrate a dental examination device with a mirror attached by a tilt mechanism with a fastening element to hold the mirror in different orientations.

### DETAILED DESCRIPTION

### Example Embodiments

The following detailed description solves one or more of the problems above. The dental examination device described below can be used by dentists, orthodontists, caregivers, or others who must exam teeth in children and patients with special needs. It allows for the common item of a toothbrush, which children and special needs patients are normally more familiar with, to be used in conjunction with a dental examination mirror to visualize the mouth and teeth. The brushing motion of the toothbrush provides a familiar distraction to the patient while the examination takes place. By combining the toothbrush and mirror in one device, the dental examination device can be used by a single user (e.g., dentist, orthodontist, or caregiver) instead of requiring one person to use the mirror and a second person to use the toothbrush.

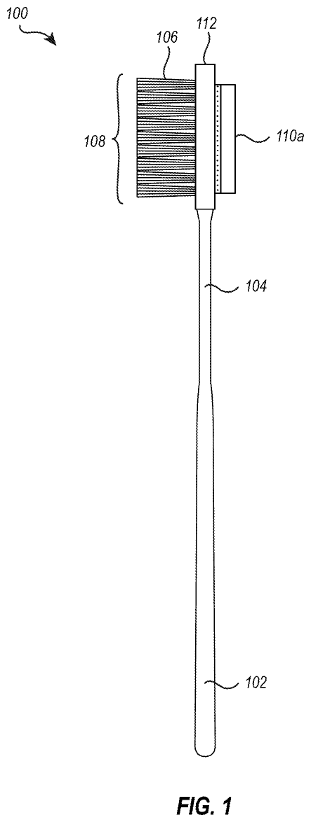

FIG. 1 illustrates an example of a dental examination device 100 which includes a handle 102, a shank 104, and a head 112. The handle 102, shank 104, and head 112 form a standard toothbrush as are well known in the art.

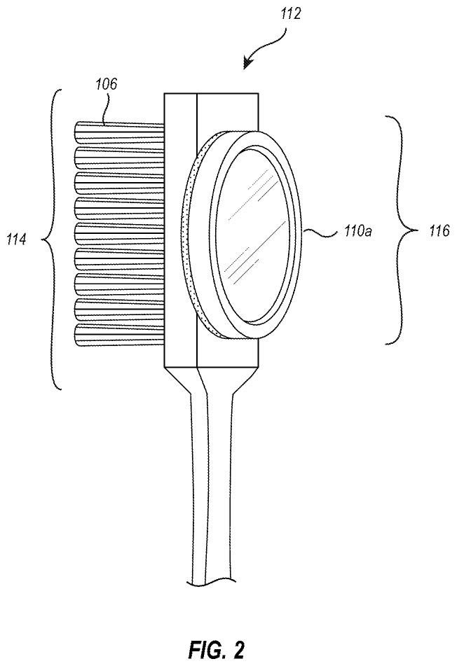

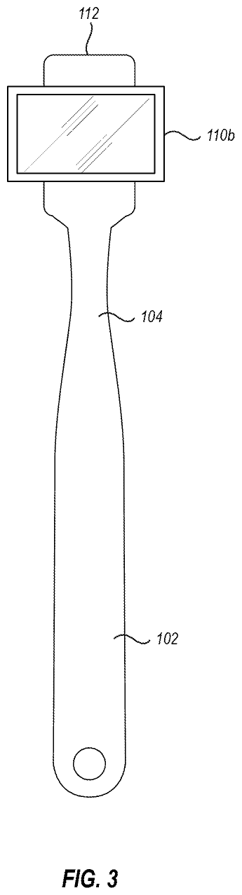

FIG. 2 illustrates a close-up view of the head which includes a front surface 114 and back surface 116. The front surface 114 has a plurality of bristles 106 which together define a brushing surface 108. The bristles 106 may also be referred to herein as tufts or other synonymous terms found in the art. The back surface 116 is coupled to a mirror 110a. In this embodiment, the mirror 110a is rounded with no edges, however, other embodiments may include mirrors with other shapes such as rounded or polygonal shapes. For example, FIG. 3 shows a polygonal mirror 110b, in particular a rectangular mirror 110b.

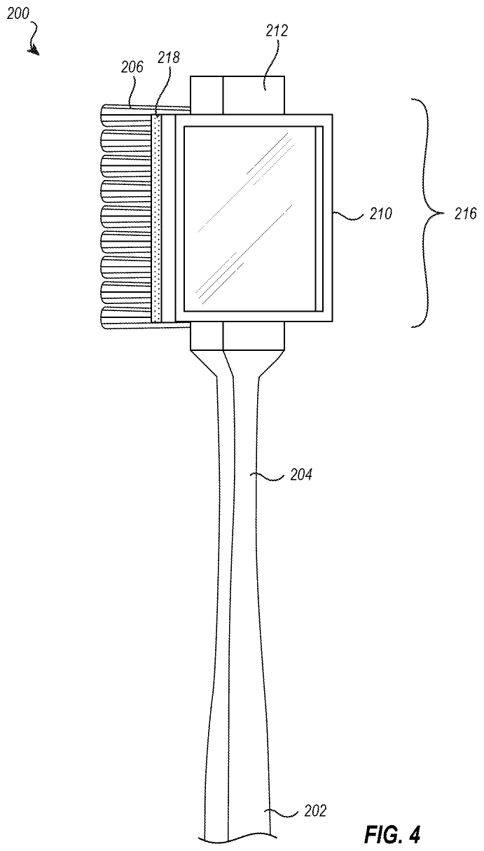

FIG. 4 illustrates another embodiment of a dental examination device 200 with features similar to dental examination device 100, except where specified otherwise, where like components have like numbers. The dental examination device 200 includes a handle 202, a shank 204, and a head 212. The head 212 includes bristles 206 and a mirror 210. In this embodiment, the mirror 210 is coupled to the back surface 216 of the head 212 using an adhesive 218. The adhesive 218 may include glue, tape, or other adhesives known in the art suitable for coupling the mirror 210 to a surface such as back surface 216.

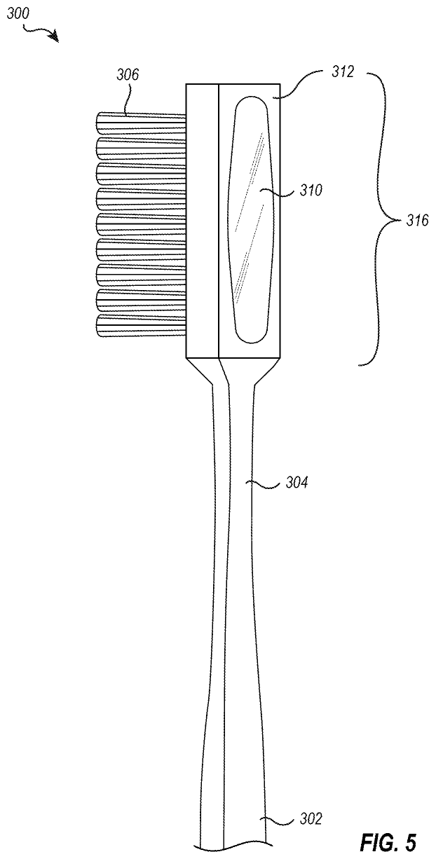

FIG. 5 illustrates another embodiment of a dental examination device 300 with features similar to the other dental examination devices described herein, except where specified otherwise, where like components have like numbers. The dental examination device 300 includes a handle 302, a shank 304, and a head 312 with a mirror 310. FIG. 5 shows a back view of the dental examination device 300 illustrating the mirror 310 embedded into the head 312. For example, one or more edge portions of the mirror 310 may be molded into the head 312 during manufacture of the dental examination device 300 such that the mirror 310 is held in place by an overmolded portion of the head 312 (i.e., a portion of the head 312 that extends over the one or more edge portions of the mirror 310). The overmolded portion of the head 312 thereby functions to maintain the mirror 310 in the desired position on the back surface 316 of the head.

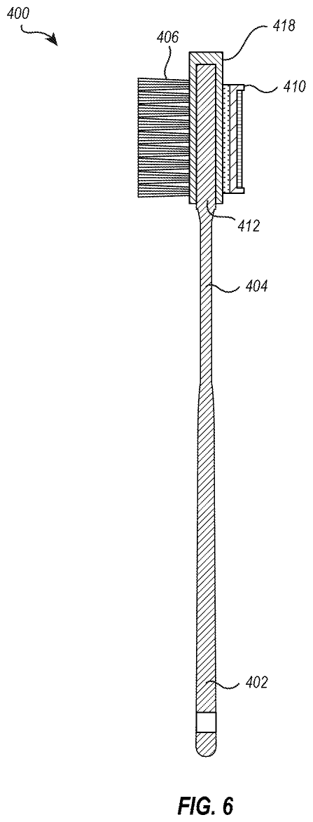

FIG. 6 illustrates another embodiment of a dental examination device 400 with features similar to the other dental examination devices described herein, except where specified otherwise, where like components have like numbers. The dental examination device 400 includes a handle 402, a shank 404, a head 412, a plurality of bristles 406, and a mirror 410. In this embodiment, the mirror 410 is selectively detachable from the head 412 using a friction fit cap 418. The friction fit cap 418 is beneficially configured to keep the bristles 406 exposed. The bristles must stay exposed because the brushing action distracts the patient with a familiar device while the examination is occurring.

In some embodiments, the friction fit cap 418 avoids overly interfering with the brushing surface of the dental examination device 400. For example, the friction fit cap 418 may be configured such that the front portion of the cap is allowed to extend with respect to the front surface of the head no more than 50%, 40%, 30%, 20%, 10%, all the way down to 5% of the average length of the bristles, or no more than 40% of the average length of the bristles.

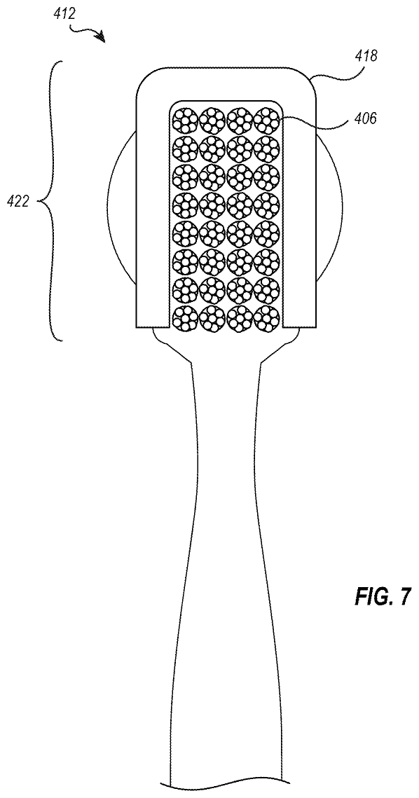

FIG. 7 illustrates a front view of the head 412 of the dental examination device 400 showing an example where the friction fit cap 418 surrounds the head 412 while keeping the bristles 406 exposed. In another embodiment, the friction fit cap 418 may only engage with the side surface 422 of the head 412 when positioned on the head 412. Other known friction fit caps in the art may be used.

As shown, the mirror 410 is attached to a back side of the friction fit cap 418. The mirror 410 may be attached to the friction fit cap 418 using any of the means described herein for attaching a mirror to the corresponding head of the toothbrush. For example, the mirror 410 may be attached to the friction fit cap 418 by an adhesive such as glue or tape or by molding the mirror into the cap.

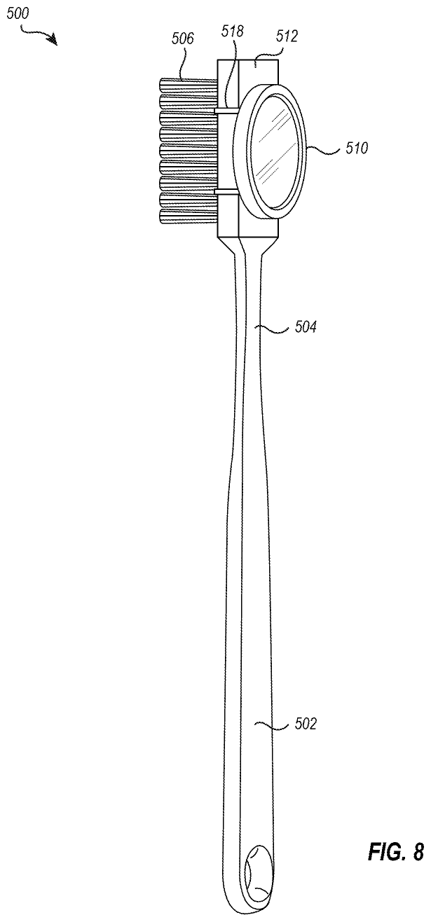

FIG. 8 illustrates another embodiment of a dental examination device 500 with features similar to the other dental examination devices described herein, except where specified otherwise, where like components have like numbers. The dental examination device 500 includes a handle 502, a shank 504, and a head 512, where the head includes bristles 506 and a mirror 510. In this embodiment, the mirror 510 is selectively detachable to the head 512 by way of an attachable element 518. In this embodiment, the attachable element 518 is configured to secure around and engage with the head 512 while still exposing the bristles 506.

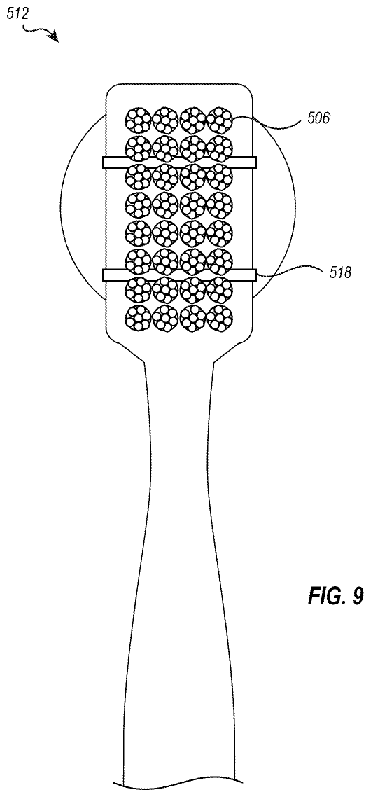

FIG. 9 illustrates a front view of an example of the dental examination device 500 where the element 518 secures around the head 512 while keeping the bristles 506 exposed. Other embodiments of element 518 may be used such as one or multiple straps securing around the head 512 or other elements known in the art suitable for securing a mirror 510 to the head 512 while still exposing the bristles 506. As shown, the attachable elements are attached to the mirror 510 and configured to wrap around (in whole or in part) the head 512 to secure the mirror 510 to the head 512. In preferred embodiments the elements are configured in size and shape to pass or weave through the bristles 506 and makes substantial contact with the front surface rather than interfere with the middle or tip portions of the bristles. This may be accomplished by the use of bands or straps that are thin enough to fit between where the bristles 506 attach to the head 512. The illustrated embodiment shows two attachable elements 518. In other embodiments, the device may utilize a single attachable element or more than two attachable elements. The attachable elements 518 may be formed from any suitable material. Examples include rubber, silicone, nylon, or polyester.

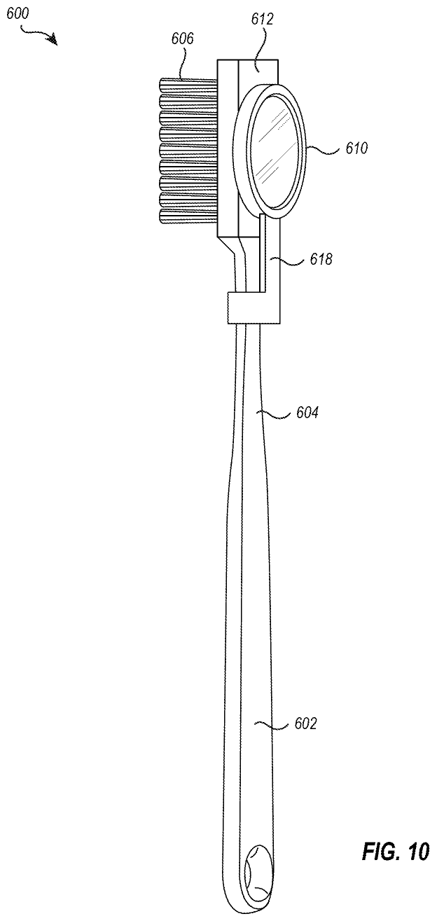

FIG. 10 illustrates another embodiment of a dental examination device 600 with features similar to the other dental examination devices described herein, except where specified otherwise, where like components have like numbers. This includes a handle 602, a shank 604, and a head 612, where the head 612 includes bristles 606 and a mirror 610. In this embodiment, the mirror 610 is selectively detachable to the head 612 by way of a hook 618 that goes around the shank 604. An example of the hook 618 is shown in FIG. 10 where the hook 618 only partially goes around the shank 604. Other embodiments may include a hook 618 that is configured to fully go around the shank 604. The hook 618 may be rigid, deformable, or resilient. The illustrated embodiment includes a single hook 618. Other embodiments may include multiple hooks. For example, an embodiment may include a series of hooks with at least one at a different longitudinal position along the shank 604 relative to at least one other hook. Additionally, or alternatively, an embodiment may include a series of hooks with one or more wrapping around one side of the shank 604 and one or more wrapping around an opposite side of the shank 604.

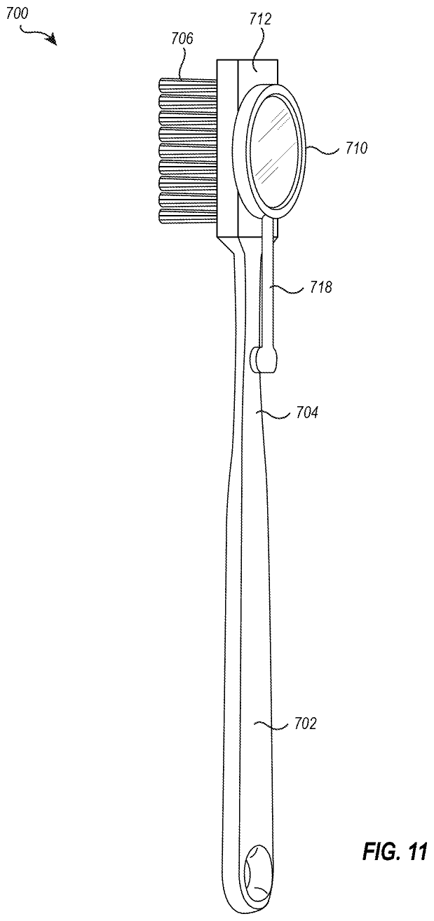

FIG. 11 illustrates another embodiment of a dental examination device 700 with features similar to the other dental examination devices described herein, except where specified otherwise, where like components have like numbers. This includes a handle 702, a shank 704, and a head 712. In this embodiment, the head 712 includes bristles 706 and a mirror 710. The mirror 710 is attached to an elongated member 718 at a first end, where an opposite second end of the elongated member 718 is attached to the handle 702. In other embodiments, the second end of the elongated member 718 may be attached to the shank 704. The elongated member 718 could be attached to the handle 702 or shank 704 by an adhesive such as glue or by molding the extended member into the handle 702 or shank 704.

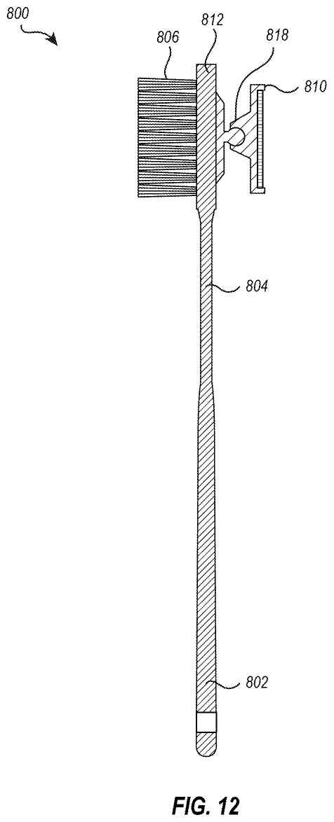

FIG. 12 illustrates another embodiment of a dental examination device 800 with features similar to the other dental examination devices described herein, except where specified otherwise, where like components have like numbers. This includes a handle 802, a shank 804, and a head 812. The head 812 includes bristles 806 and a mirror 810. The mirror 810 is attached to the head 812 by a tilt mechanism 818. The tilt mechanism 818 allows the mirror 810 to be selectively moved to a desired orientation and held in that orientation by friction or resistance. This is beneficial to the examiner because they can move the mirror into an orientation to visualize specific areas of the mouth before inserting the device into the patient's mouth. For example, if the examiner wants to visualize the upper molars, the examiner can tilt the mirror to an orientation that slightly tilts downward so when inserted into the patient's mouth, the mirror shows the entire tooth while keeping the mirror in a position that can easily be seen.

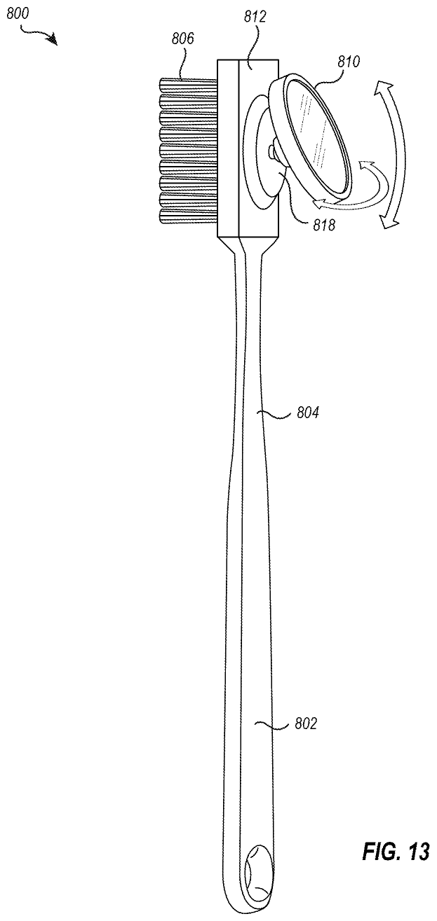

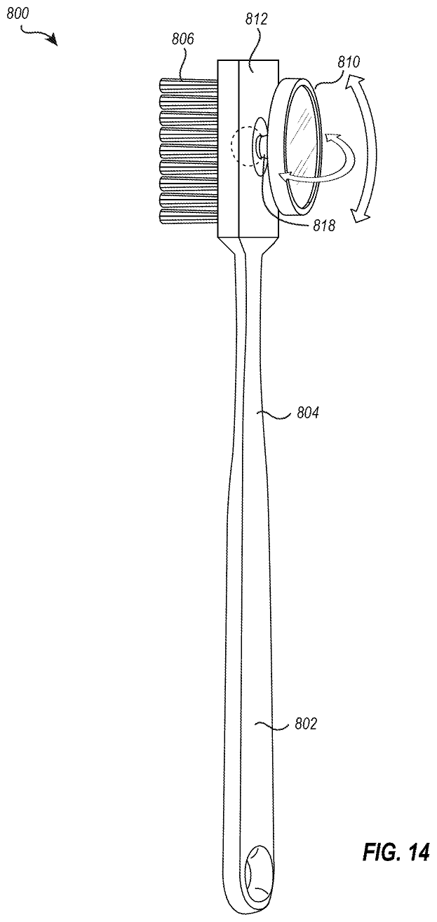

FIG. 13 illustrates an example where the mirror 810 is tilted along the longitudinal axis of the head 812 and FIG. 14 illustrates an example where the mirror 810 is tilted along the latitudinal axis of the head 812. The tilt mechanism 818 is preferably flexible/movable enough to enable the user to easily move to a particular orientation but rigid enough to then hold that orientation at least temporarily (e.g., at least during the typical time the device is within the patient's mouth). The tilt mechanism 818 may be comprised of a flexible material that can hold its shape, such as, for example, armature wire. The tilt mechanisms 818 may additionally or alternatively include a hinge, a ball and socket joint, a prismatic joint, or other joints or tilting mechanisms known in the art that are suitable to move and then hold the mirror 810 in a certain orientation.

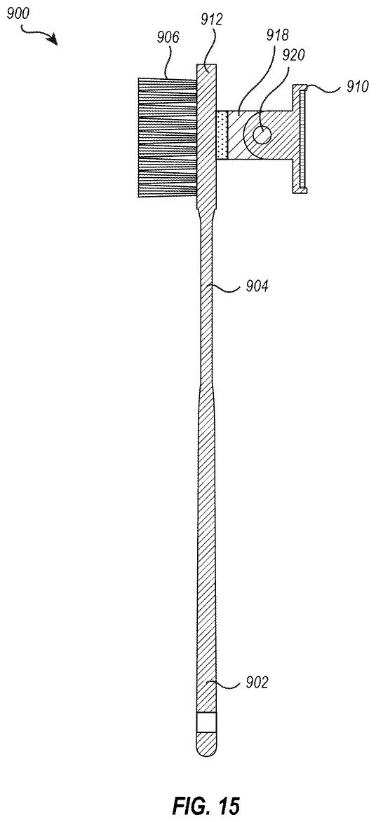

FIG. 15 illustrates another embodiment of a dental examination device 900 with features similar to the other dental examination devices described herein, except where specified otherwise, where like components have like numbers. This includes a handle 902, a shank 904, and a head 912, which includes bristles 906 and a mirror 910. The mirror is attached to the head by a tilt mechanism 918 and a fastening element 920. The tilt mechanism 918 is preferably flexible/moveable enough to move the mirror 910 in different orientations. The fastening element 920 can be loosened or unlocked to allow the mirror 910 to tilt and then tightened or locked to hold the mirror 910 in a particular orientation.

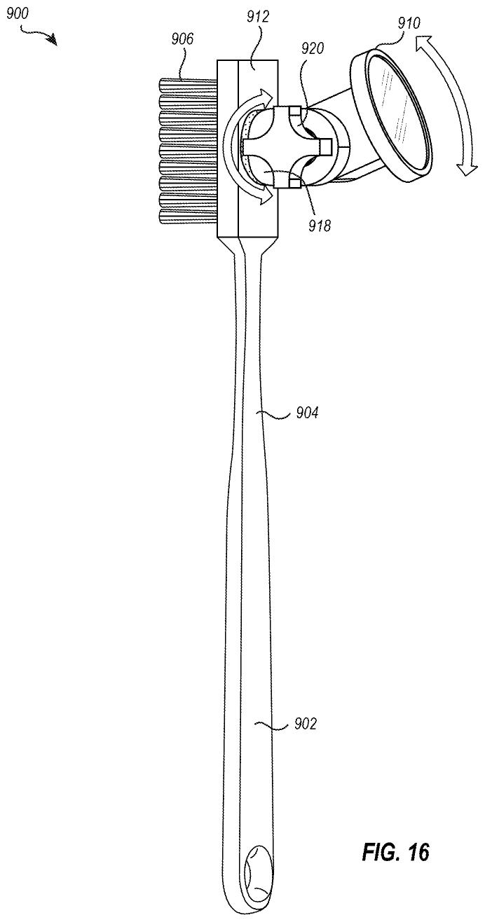

FIG. 16 shows an example of the mirror 910 held in a particular orientation by the tilt mechanism 918 and fastening element 920. The fastening element 920 may be, for example, a screw that when loosened allows the tilt mechanism 918 to move and then when tightened holds the tilt mechanism 918 in place. Other embodiments may additionally or alternatively include a fastening element 920 that pulls away from the tilt mechanism 918 to allow the tilt mechanism 918 to move and then pushes into the tilt mechanism 918 to hold the mirror 910 in a particular orientation (e.g., via a “snap” fitting arrangement). Other fastening elements 920 known in the art suitable for allowing a tilt mechanism 918 to move and then be held in place may also be used.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise. For example, the features described in relation to the embodiment of FIG. 3, including the feature of the polygonal shaped mirror 110b may be combined with the features described in relation to other embodiments of the dental examination device (e.g. device 500 shown in FIG. 8). Another example is the features described in relation to the embodiment of FIG. 12, including the feature of the tilt mechanism 818 may be combined with the features described in relation to the friction fit cap 418 illustrated in FIG. 6.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A dental examination device, comprising:
a handle attached to a shank attached to a head, the head having a front and back surface;
a plurality of bristles extending from the front surface of the head, defining a brushing surface; and
a mirror coupled to the back surface of the head.

2. The dental examination device of claim 1, wherein the mirror has a polygonal shape.

3. The dental examination device of claim 2, wherein the polygonal shape is rectangular.

4. The dental examination device of claim 1, wherein the mirror has no edges.

5. The dental examination device of claim 4, wherein the mirror is circular, oval, or a rounded shaped.

6. The dental examination device of claim 1, wherein the mirror is integrated to the back surface of the head.

7. The dental examination device of claim 6, wherein the mirror is embedded in the back surface of the head.

8. The dental examination device of claim 6, wherein the mirror is attached using an adhesive to the back surface of the head.

9. The dental examination device of claim 1, wherein the mirror is coupled to the back surface of the head by an elongated member attached on one end to the mirror and on the other end to the shank or handle.

10. The dental examination device of claim 1, wherein the mirror is selectively detachable from the back surface of the head.

11. The dental examination device of claim 7, wherein the mirror is attached to the back surface of the head by a hook configured to engage around the shank.

12. The dental examination device of claim 7, wherein the mirror is attached to the back surface of the head by a friction fit cap.

13. The dental examination device of claim 7, wherein the mirror is attached to an attachable element that is configured to secure around the head while keeping the bristles exposed.

14. The dental examination device of claim 1, wherein the mirror is attached to the back surface by a tilt mechanism.

15. The dental examination device of claim 14, wherein the tilt mechanism provides movement of the mirror relative to the back surface.

16. The dental examination device of claim 14, wherein the tilt mechanism holds the mirror in a particular orientation using resistance.

17. The dental examination device of claim 14, wherein the tilt mechanism holds the mirror in a particular orientation by a fastening element.

18. A method for examining a patient's mouth, comprising:
providing the dental examination device of claim 1;
placing the dental examination device in the patient's mouth;
brushing the patient's teeth with the brushing surface; and
visualizing the patient's teeth with the mirror.
